(19) United States
(12) Patent Application Publication
(10) Pub. No.: US 2025/0116626 A1
(43) Pub. Date: Apr. 10, 2025

(54) Electrochemical Aptamer Sensors with Signal Amplification Via Multiple Redox Tags

(71) Applicant: University of Cincinnati, Cincinnati, OH (US)

(72) Inventors: Jason Heikenfeld, Cincinnati, OH (US); Mark Friedel, Cincinnati, OH (US)

(73) Assignee: University of Cincinnati, Cincinnati, OH (US)

(21) Appl. No.: 18/728,021

(22) PCT Filed: Jan. 10, 2023
(86) PCT No.: PCT/US23/10471

§ 371 Date: Jul. 10, 2024

Related U.S. Application Data
(60) Provisional application No. 63/299,084, filed on Jan. 13, 2022.

(51) Int. Cl. G01N 27/327; G01N 33/53; G01N 33/543

(52) U.S. Cl. CPC G01N 27/3277; G01N 27/3276; G01N 33/5308; G01N 33/5438

(57) ABSTRACT

A device for detecting the presence of, or measuring the concentration or amount of, at least one analyte in a sample fluid is disclosed. The device includes at least one electrode; a sample fluid; and a plurality of affinity-based probes capable of binding to the analyte, wherein the affinity-based probes each carry a plurality of redox tags. Further, the detection or measurement of an analyte is caused by analyte binding to the affinity-based probe which further causes a change in electron transfer from the redox tags.

### CROSS-REFERENCE TO RELATED APPLICATIONS

This application is a continuation of PCT Application No. PCT/US23/10471 filed Jan. 10, 2023, which claims the benefit of the filing date of U.S. Provisional Application No. 63/299,084 filed Jan. 13, 2022, the disclosures of which are incorporated by reference herein in their entireties.

### FIELD OF THE INVENTION

This invention relates generally to aptamer sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox tag) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of concentration of the analyte. Aptamers are an example of an affinity-based biosensor.

A major unresolved challenge for aptamer sensors and other affinity-based biosensors (particularly those where the aptamers are bonded to the working electrode) is the lifetime of the sensors, especially for applications where continuous operation is required (“continuous” referring to multiple measurements over time by the same device). Such aptamer sensors are susceptible to degradation due to, among other things, desorption from the electrode of the aptamers themselves and/or the blocking molecules such as mercaptohexanol. The aptamers and the blocking molecules together for a monolayer which can be referred to as a sensing monolayer. The blocking layer portion of the sensing monolayer is critical to ensure the aptamer switching with analyte binding moves freely and properly, and for reducing electrical background current (which includes oxygen reduction current) which would otherwise wash-out the measured signal from the aptamer and redox tag.

There are several ways to begin to address the issue of aptamer longevity, for example including blocking layers with multiple thiol linkages, or by relying on natural passivation of the electrode with serum solutes from the sample fluid. However, many such techniques increase the electrochemical background current compared to a fragile, but low background current, blocking layer of mercaptohexanol. Furthermore, in some designs, regardless of longevity issues, signal strength vs. background noise is inadequate. Therefore devices, materials, and methods are needed to increase the signal strength vs. background noise in aptamer and other affinity-based electrochemical sensors.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to device for detecting the presence of, or measuring the concentration or amount of, at least one analyte in a sample fluid. The device includes at least one electrode; a sample fluid; and a plurality of affinity-based probes capable of binding to the analyte, wherein the affinity-based probes each carry a plurality of redox tags. Further, the detection or measurement of an analyte is caused by analyte binding to the affinity-based probe which further causes a change in electron transfer from the redox tags. In one embodiment, the affinity-based probe is an aptamer. In another embodiment, the aptamer is bound to the electrode or a material that itself is directly or indirectly attached to the electrode. In one embodiment, the aptamer is in solution.

In another embodiment, the device has a redox signal strength, and the redox signal strength, when compared to a probe with a singular redox tag, is at least 1.5 times greater. In one embodiment, the device has a redox signal strength, and the redox signal strength, when compared to a probe with a singular redox tag, is at least 2 times greater. In another embodiment, the device has a redox signal strength, and the redox signal strength, when compared to a probe with a singular redox tag, is at least 3 times greater. In one embodiment, the device has a redox signal strength, and the redox signal strength, when compared to a probe with a singular redox tag, is at least 10 times greater.

In another embodiment, the redox tags are the same molecule. In one embodiment, each redox tag comprises at least one molecule that is distinct from the other redox tags. In another embodiment, the redox tags have an average position relative to the electrode during measurement, and the redox tags with further position from the electrode have a redox peak voltage when free in solution that is less than the redox peak voltage for redox tags in closer position to the electrode. In one embodiment, the redox tags contributing to the measured signal each have a redox potential measured by the electrode and the redox potentials are within 0.125 V or less of each other. In another embodiment, the affinity-based probes each carry at least three redox tags.

In another aspect of the present invention, a method of detecting or measuring at least one analyte in a sample fluid is provided. The method involves placing a device in a sample fluid, the sample fluid having at least one analyte. The device includes at least one electrode and a plurality of affinity-based probes capable of binding to the analyte. Further, the affinity-based probes each carry a plurality of redox tags. The method additionally involves detecting or measuring the analyte, where the detection or measurement of the analyte is caused by analyte binding to the affinity-based probe which further causes a change in electron transfer from the redox tags. In one embodiment, the affinity-based probe used in the method is an aptamer.

In another embodiment, the device used in the method has a redox signal strength, and the redox signal strength, when compared to a probe with a singular redox tag, is at least 1.5 times greater. In one embodiment, the device used in the method has a redox signal strength, and the redox signal strength, when compared to a probe with a singular redox tag, is at least 3 times greater. In another embodiment, the device used in the method has a redox signal strength, and the redox signal strength, when compared to a probe with a singular redox tag, is at least 10 times greater.

In one embodiment, the redox tags used in the method comprise the same molecule. In another embodiment, each redox tag used in the method comprises at least one molecule that is distinct from the other redox tags.

### BRIEF DESCRIPTION OF THE DRAWINGS

The objects and advantages of the disclosed invention will be further appreciated in light of the following detailed descriptions and drawings in which:

FIG. 1 is a schematic of a conventional prior art sensor device.

FIG. 2 is a schematic of a device of an embodiment of the present invention.

FIG. 3A is a schematic showing a traditional (single-tagged) aptamer with unaffected binding region or conformation.

FIG. 3B is a schematic showing a triple-tagged aptamer according to the present invention with unaffected binding region or conformation.

FIG. 4 is a graph of square wave voltammograms showing the 5.4× increase in signal produced by triple-tagged aptamers in buffer with shaded error regions.

FIG. 5A is a graph showing the signal gain of single-tagged phenylalanine aptamers across a range of 10 nM-10 mM target at signal off (10 Hz) and signal on (30-300 Hz) frequencies.

FIG. 5B is a graph showing the signal gain of triple-tagged phenylalanine aptamers across a range of 10 nM-10 mM target at signal off (10 Hz) and signal on (30-300 Hz) frequencies.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “electrode” means any material that is electrically conductive such as gold, platinum, nickel, silicon, conductive liquid infused materials such as ionic liquids, PEDOT:PSS, conductive oxides, carbon, boron-doped diamond, nanotubes or nanowire meshes, or other suitable electrically conducting materials.

As used herein, the term “blocking layer” means a homogeneous or heterogeneous layer of material or of one or more types of molecules on an electrode which reduce electrochemical background current and/or current due to electrochemical interference, and which may promote proper freedom of movement for the aptamer which is required for creating a measurable response to analyte concentration.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, proteins, and affimers. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “redox tag” or “redox molecule” means any species such as small or large molecules with a redox active portion that when brought adjacent to an electrode can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a redox mediator. Redox tags or molecules may also exchange electrons or change in behavior when brought into proximity with other redox tags or molecules.

As used herein, the term “change in electron transfer” means a redox molecule whose electron transfer with an electrode has changed in a measurable manner. This change in electron transfer can, for example, originate from availability for electron transfer, distance from an electrode, diffusion rate to or from an electrode, a shift or increase or decrease in electrochemical activity of the redox molecule, or any other embodiment as taught herein that results in a measurable change in electron transfer between the redox molecule and the electrode.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “continuous sensing” simply means the device records a plurality of readings over time. Even a point-of-care testing device which provides a single data point can be considered a continuous sensing device if, for example, it is a 15 minute test, that operates by taking multiple data points over 15 minutes and averaging them to provide a single data measure.

As used herein, a “device” comprises at least one sensor based on at least one aptamer, at least one sensor solution, and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a reference or counter electrode, a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

In one embodiment, the present invention involves attaching a plurality of redox tags to an affinity-based probe to increase the signal strength vs. background noise in aptamer and other affinity-based electrochemical sensors.

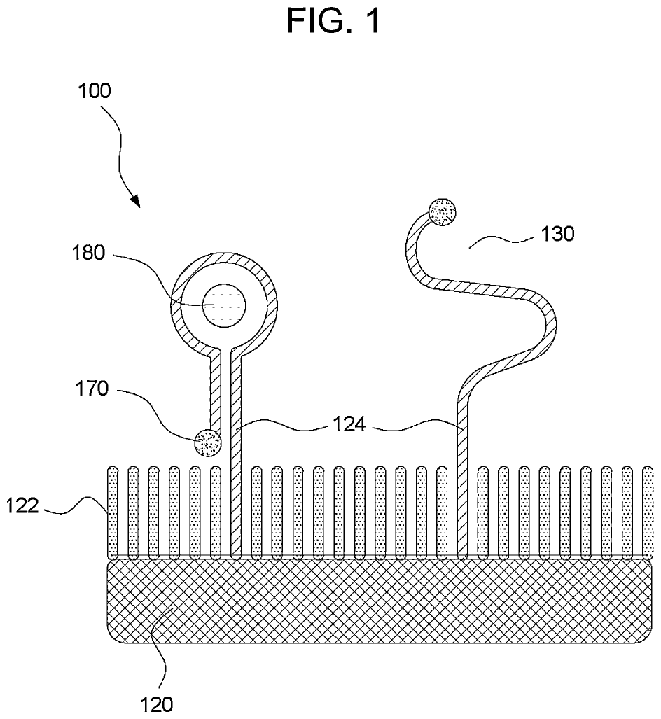

With reference to FIG. 1, a conventional prior art sensor device 100 as placed initially in a sample fluid 130, such as interstitial fluid, is shown. The sensor comprises: at least one working electrode 120 such as gold, carbon, or other suitable electrode material; at least one blocking layer 122 of a plurality of molecules such as mercaptohexanol or hexanethiol that are thiol bonded to the electrode, or a plurality of natural solutes in blood that can act as a blocking layer, or other suitable molecules depending on application and on the choice of electrode 120 material; at least one aptamer 124 that is responsive to binding to an analyte 180 and which contains a redox tag 170 such as methylene blue. In the generic example taught for FIG. 1, the aptamer 124 is a simple stem loop (hairpin) aptamer where analyte 180 binding causes the stem loop to form and the redox current measured from the redox tag 170 to increase, as measured using square wave voltammetry or other suitable technique. In absence of analyte 180 binding to the aptamer 124 the stem loop is broken and the redox current would decrease. Thus a measurement of electrical current can be used to interpret changes in concentration of the analyte 180. A device such as FIG. 1, will eventually degrade as the blocking layer 122 desorbs, increasing background current, such that the redox tag 170 signal change due to analyte 180 binding has greater error due to background noise or is even no longer measurable.

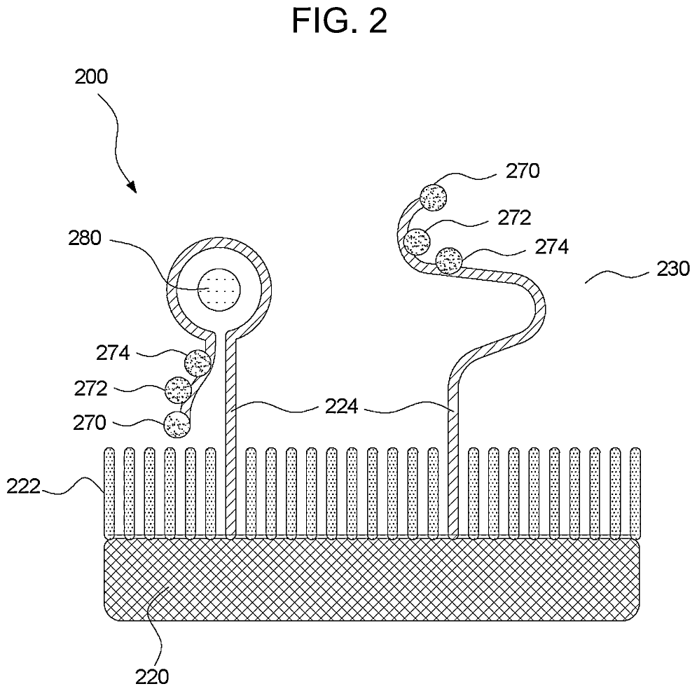

With reference to FIG. 2, where like numerals refer to like features, a device 200 has a plurality of redox tags 270, 272, 274. For example, consider an aptamer like that of FIG. 1 where the background current was 100 nA and the redox peak current plus baseline current was 150 nA (50% above background current), and after 24 hours the background current increased to 500 nA (redox peak only 10% of background current, too much noise compared to signal). With the aptamer of FIG. 2, with, for example, 3 redox tags 270, 272, 274, the redox peak current plus baseline could instead be 250 nA (150% of background current), and after 24 hours the redox peak current will still be 50% greater than background current, resulting in an improved or more readable signal change with analyte 280 binding. Although the approach of a plurality of redox tags does not necessarily improve signal gain (% change in the peak) it does improve signal strength which can then improve device 200 measurement accuracy and/or longevity. The present invention is capable of 2, 3, 5, or even 10 redox tags, or more, improving redox signal strength compared to a singularly tagged aptamer by at least one of 1.5×, 2×, 3×, 10×.

With further reference to embodiments of the present invention, a plurality of redox tags, for example, can be attached along thymine groups in the aptamer as needed, or using other suitable techniques, such as attaching a molecule or nanoparticle to the aptamer and further attaching a plurality of redox tags to that molecule or nanoparticle. Generally, optimal performance is achieved if the redox tags are all tagged in a similar region of the aptamer, else they could cancel each other out in terms of signal change with aptamer shape change due to analyte binding. Furthermore, if the redox tags are too far apart in distance, it can result in broadening of the measured redox peak with respect to scanned voltage. A plurality of redox tags can also be useful, for example, where signal strength is too weak, for example when measuring aptamers with square wave voltammogram signals that are measured at <10's of Hz. To result in increased signal strength compared to background current, generally the redox tags (methylene blue, ferrocene, anthraquinones, etc.) should have the same redox potential such that their redox peaks overlap. Alternately, one or more of the plurality of redox tags could be a different redox tag, a redox tag with a distinct local chemical environment, or other suitable technique which helps improve redox tag peak overlap during measurement. For example, a first redox tag that on average is brought closer to the electrode could have first redox peak potential (as measured free in solution) that is slightly larger in voltage than a second redox tag that on average is more distant from the electrode and that would have a second redox peak potential (as measured free in solution) that is slightly smaller in voltage that the first redox peak, because redox peaks are shifted slightly to higher potentials with distance of the redox tag from the electrode. In one embodiment, the redox tags contributing to the measured signal will each have respective redox peaks as measured by the electrode that are within 0.125V or less of each other. Redox tags can also exchange redox state with each other, such that if a first redox tag closest to the electrode is reduced or oxidized, other redox tags in adjacency can then either in parallel or series reaction can be reduced or oxidized.

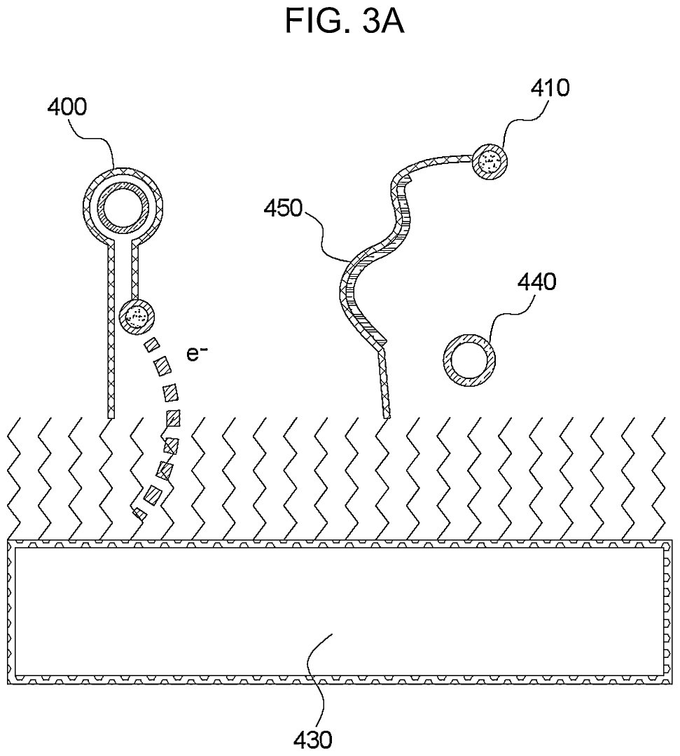

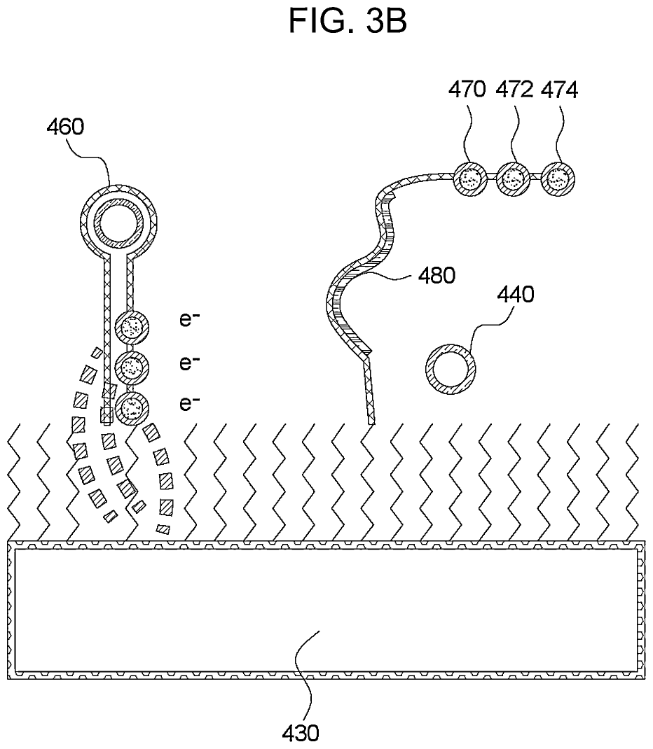

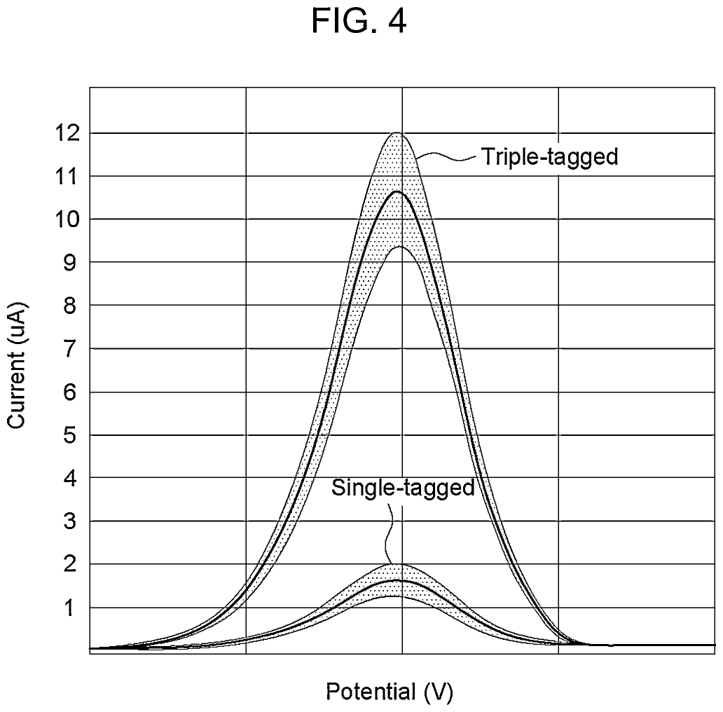

With reference to FIG. 3A, a schematic shows a traditional aptamer 400 with a single tag 410 attached to a gold electrode 430 in a solution with an analyte 440. The aptamer 400 has a binding region 450. FIG. 3B shows an aptamer 460 with three tags 470, 472 and 474. The aptamer 460 is attached to a gold electrode 430 in a solution with an analyte 440. The aptamer 460 has a binding region 480. The “triple-tagged” aptamer 460 shows increased response when compared to traditional aptamer 400 (see FIG. 4).

### EXAMPLES

### Example 1

With reference to FIG. 4, a graph of square wave voltammograms shows the 5.4× increase in signal produced by triple-tagged aptamers in buffer with shaded error regions. Data was collected using standard (2 mm diameter) gold disc electrodes prepared with ‘typical or single-tagged’ or ‘triple-tagged’ aptamer and then insulated with 6-mercapto-1-hexanol following common procedures (a 1-hour incubation in 400 nanomolar aptamer solution followed by 2-hour incubation in 5 mM 6-mercapto-1-hexanol). After preparation functional electrodes (sensors) were placed in testing buffer (Phosphate buffer saline) and connected to a potentiostat. Measurements were taken using square wave voltammetry with the following parameters: a 10 Hz or 300 Hz pulse frequency, 35 mV pulse amplitude, and a 1 mV step increment.

Aptamers used in the examples included SEQ. 1:

SEQ. 1:
CGACC-GCGTT-TCCCA-AGAAA-GCAAG-TATTG-GTTGG-TCG

The aptamers were either a single tag 1×MB-L-Phe Aptamer as follows:

/5ThioMC6-D/ SEQ. 1 /3MeBlN/

Or a triple tagged 3×MB-L-Phe aptamer as follows:

/5ThioMC6-D/ SEQ. 1 /iMeBlN/TTT/iMeBlN/TT /3MeBlN/

Wherein /iMeBlN/=internal methylene blue, and /TTT/ is a 3 base pair spacer.

### Example 2

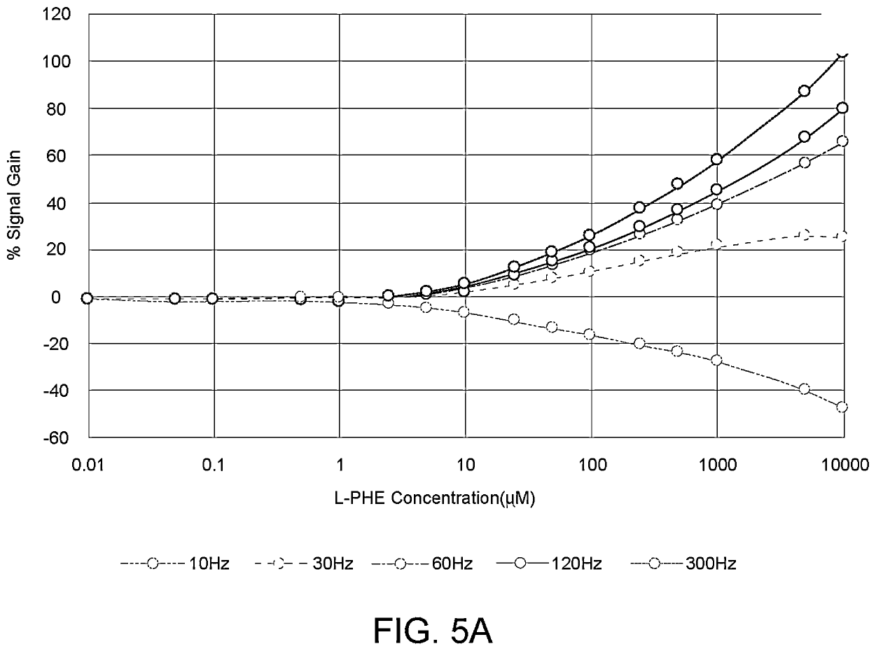

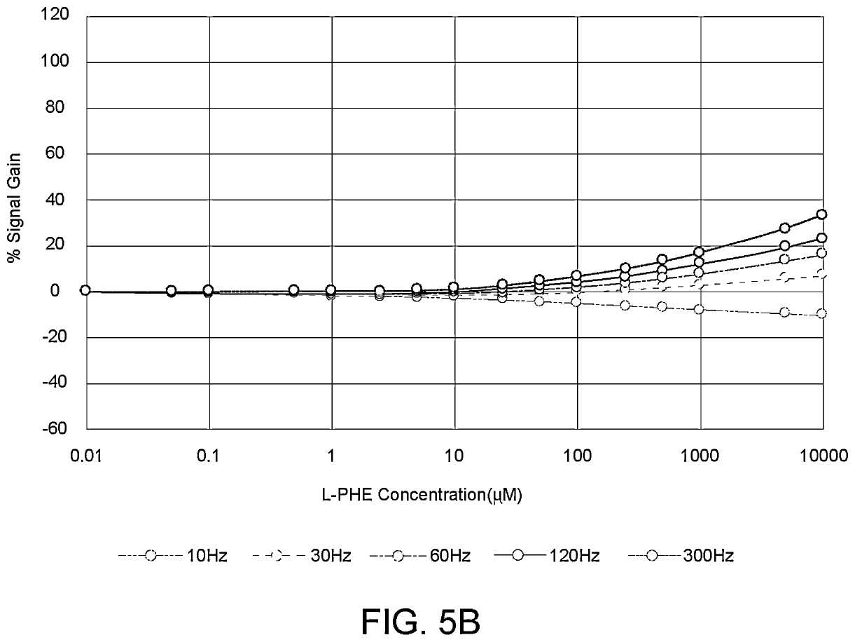

With reference to FIG. 5A, a graph shows the signal gain of single-tagged phenylalanine aptamers across a range of 10 nM-10 mM target at signal off (10 Hz) and signal on (30-300 Hz) frequencies. Electrodes were prepared and data was collected following the same procedures and square wave voltammetry parameters as those described in Example 1. ‘Typical’ or ‘single-tagged’ aptamers we measured at pulse frequencies ranging from 10 to 300 Hz. Phenylalanine solution was titrated into testing buffer and stirred before each measurement, testing various concentrations between 10 nM and 10 mM. A baseline measurement used to calculate gain was taken in solution free of phenylalanine. With reference to FIG. 5B, a graph shows the signal gain of triple-tagged phenylalanine aptamers across a range of 10 nM-10 mM target at signal off (10 Hz) and signal on (30-300 Hz) frequencies. Electrodes were prepared and data was collected following the same procedures and square wave voltammetry parameters as those described above, with the exception that sensors used to form FIG. 5B were created using ‘triple-tagged’ phenylalanine aptamers.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for detecting or measuring at least one analyte in a sample fluid, the device comprising:
at least one electrode;
a sample fluid; and
a plurality of affinity-based probes capable of binding to the analyte, wherein the affinity-based probes each carry a plurality of redox tags;
wherein the detection or measurement of an analyte is caused by analyte binding to the affinity-based probe which further causes a change in electron transfer from the redox tags.

2. The device of claim 1, wherein the affinity-based probe is an aptamer.

3. The device of claim 1, wherein the device has a redox signal strength, and the redox signal strength, when compared to a probe with a singular redox tag, is at least 1.5 times greater.

4. The device of claim 1, wherein the device has a redox signal strength, and the redox signal strength, when compared to a probe with a singular redox tag, is at least 2 times greater.

5. The device of claim 1, wherein the device has a redox signal strength, and the redox signal strength, when compared to a probe with a singular redox tag, is at least 3 times greater.

6. The device of claim 1, wherein the device has a redox signal strength, and the redox signal strength, when compared to a probe with a singular redox tag, is at least 10 times greater.

7. The device of claim 1, wherein the redox tags comprise the same molecule.

8. The device of claim 1, wherein each redox tag comprises at least one molecule that is distinct from the other redox tags.

9. The device of claim 1, wherein the redox tags have an average position relative to the electrode during measurement, and the redox tags with further position from the electrode have a redox peak voltage when free in solution that is less than the redox peak voltage for redox tags in closer position to the electrode.

10. The device of claim 1, wherein the redox tags contributing to the measured signal each have a redox potential measured by the electrode and the redox potentials are within 0.125 V or less of each other.

11. The device of claim 2, wherein the aptamer is bound to the electrode or a material that itself is directly or indirectly attached to the electrode.

12. The device of claim 2, wherein the aptamer is in solution.

13. The device of claim 1 wherein the affinity-based probes each carry at least three redox tags.

14. A method of detecting or measuring at least one analyte in a sample fluid, the method comprising:
a. placing a device in a sample fluid, the sample fluid comprising at least one analyte, wherein the device comprises at least one electrode and a plurality of affinity-based probes capable of binding to the analyte, the affinity-based probes each carrying a plurality of redox tags; and
b. detecting or measuring the analyte;
wherein the detection or measurement of the analyte is caused by analyte binding to the affinity-based probe which further causes a change in electron transfer from the redox tags.

15. The method of claim 14 wherein the affinity-based probe is an aptamer.

16. The method of claim 14 wherein the device has a redox signal strength, and the redox signal strength, when compared to a probe with a singular redox tag, is at least 1.5 times greater.

17. The method of claim 14 wherein the device has a redox signal strength, and the redox signal strength, when compared to a probe with a singular redox tag, is at least 3 times greater.

18. The method of claim 14 wherein the device has a redox signal strength, and the redox signal strength, when compared to a probe with a singular redox tag, is at least 10 times greater.

19. The method of claim 14 wherein the redox tags comprise the same molecule.

20. The method of claim 14 wherein each redox tag comprises at least one molecule that is distinct from the other redox tags.


SEQUENCE LISTING (1 sequence)

SEQ ID NO: 1 - DNA, 38 bp, synthetic construct
  source 1..38: mol_type = other DNA
cgaccgcgtttcccaagaaagcaagtattggttggtcg